(19) United States
(12) Patent Application Publication
(10) Pub. No.: US 2005/0143741 A1
(43) Pub. Date: Jun. 30, 2005

(54) Device for introducing a suture thread anchor into a bone

(76) Inventors: Andre Timmermans, Ruurlo (NL); Georg Lajtai, Althofen (AT)

Correspondence Address: ST. ONGE STEWARD JOHNSTON & REENS, LLC, 986 BEDFORD STREET, STAMFORD, CT, 06905-5619, US

(21) Appl. No.: 10/810,288

(22) Filed: Mar. 26, 2004

Related U.S. Application Data
(63) Continuation of application No. PCT/EP02/10298, filed on Sep. 13, 2002.

(30) Foreign Application Priority Data
Sep. 26, 2001 (DE) 101 49 396.7

(51) Int. Cl. A61F 2/30

(52) U.S. Cl. 606/70

(57) ABSTRACT

A device for introducing a suture thread anchor into a bone has a hollow guide sleeve through which the suture thread anchor can be passed. The distal end of the guide sleeve is provided with a contour which makes positioning of the distal end easier. The contour is a point projecting distally from a front annular face of the guide sleeve. A remaining part of the annular face forms a shoulder with the point.

### CROSS REFERENCE OF PENDING APPLICATION

This application is a continuation of pending international patent application PCT/EP02/10298 filed on Sep. 13, 2002, which designates the United States, and which claims priority of German patent application 101 49 396.7 filed on Sep. 26, 2001.

### BACKGROUND OF THE INVENTION

The invention relates to a device for introducing a suture thread anchor into a bone, with a hollow guide sleeve through which the suture thread anchor can be passed, the distal end of the guide sleeve being provided with a contour which makes positioning of the distal end easier.

Such a device is known from U.S. Pat. No. 5,951,559.

A suture thread anchor is inserted into a bone so that a tissue which has become detached from said bone, for example a tendon, a cartilage or a bone part, can be reattached. The suture thread anchor is driven into the bone, the proximal end of said suture thread anchor having an eyelet through which a suture thread is engaged. After the suture thread anchor has been introduced into the bone, the tendon detached from the bone is reapplied to the bone by knotting the suture thread.

A typical indication for the use of such suture thread anchors includes shoulder injuries and instabilities.

The socket or glenoid cavity in which the head of the humerus sits is surrounded by a raised, crater-like margin of cartilage and tendinous tissue. The biceps tendon is also attached to this glenoid margin.

Traumatic detachment of the upper part of the labrum from the glenoid margin, with involvement of the point of insertion of the long biceps tendon, is known as a Bankart lesion.

Various mechanisms of injury are implicated. In the case of extreme traction on the long biceps tendon, for example at the end of a throwing action, the braking process, with the elbow at the same time fully extended, may cause damage to the labrum. Falling on the elbow when the arm is flexed, with upward subluxation of the head of the humerus, can cause proximal displacement of the upper part of the labrum and the long biceps tendon.

In the surgical technique mentioned at the outset, the cartilage or bone part or tendon parts that have become detached from the glenoid margin are fixed in place again by introduction of a suture thread anchor whose suture thread is used to fix the tendon in place again.

In the surgical technique described in abovementioned U.S. Pat. No. 5,951,559, the suture thread anchor is fitted at the distal end of a suture thread rotator, which is a kind of screwdriver, and pushed through a hollow guide sleeve. In the actual operation, this hollow guide sleeve is introduced into the shoulder joint by way of an arthroscopic portal and attached to the glenoid margin. For easier and secure positioning, a contour is provided at the distal end of the guide sleeve.

In abovementioned U.S. Pat. No. 5,951,559, this contour is in the shape of a two-pronged fork which is configured in such a way that it can be applied firmly and with a good fit on the raised, crater-like margin of the glenoid cavity. The necessary manoeuvres can then be performed via the guide sleeve which has been thus applied. First, a drill can be guided through it in order to drill the holes for the anchor, and then the combination of suture thread anchor and suture thread rotator is passed through it and the anchor turned in. Then, using other instruments, the suture thread protruding proximally from the anchor is threaded round the detached glenoid margin and knotted to the latter in order to reattach it on the glenoid cavity.

From U.S. Pat. No. 5,690,677 it is already known to additionally bevel the guide sleeve so as to produce a blade-like or scalpel-like point. This point, turned approximately 90° about the longitudinal axis, is additionally provided with the fork-like projection in order to fit on the glenoid margin.

The disadvantage of the aforementioned constructions is that although the sleeve can be fitted securely on the glenoid margin, it is sometimes desirable, because of the lesion or because of anatomical circumstances, to fit the suture thread anchor not directly under the glenoid margin, but instead laterally offset or tilted.

It is therefore an object of the present invention to develop a device of the type mentioned at the outset in such a way that a suture thread anchor can be introduced in a great many positions, including positions offset or tilted in relation to the glenoid margin, but while at the same time ensuring a secure fit of the guide sleeve.

### SUMMARY OF THE INVENTION

According to the invention, the object is achieved by the fact that the contour is designed as a single point projecting distally from a front annular face of the guide sleeve, and that the remaining part of the annular face forms a shoulder with the point.

By providing a shoulder and only one point, it is now possible for the distal end of the guide sleeve to be applied with great versatility, but still with a secure fit. This results in a considerable gain in the freedom of positioning, i.e. an applied sleeve can be tilted in several directions, while the shoulder between annular face and point still ensures a secure fit.

Depending on how the point is designed, it can also be used to search for a preliminary point of attachment, simply by pushing the point into the cartilage or tendinous tissue, and thereafter to select, possibly with endoscopic monitoring, a suitable alignment or orientation. Once this has been done, the guide sleeve can be pushed in until it is resting via its shoulder and thereby ensures the secure fit. Thus, the number of possible manoeuvres has been increased while at the same time maintaining a secure fit.

In a further embodiment of the invention, the annular face extends approximately perpendicular to the longitudinal axis of the guide sleeve.

This measure has the advantage that the geometry involved provides the operating surgeon with additional aids to orientation and application, and, depending on the design of the point, a more or less sharply defined shoulder of lesser or greater surface area is present for positioning the guide sleeve.

In a further embodiment of the invention, the point projects from over a circumferential area of the front annular face.

This measure has the advantage that the greater the circumferential area, the more the point can serve as an additional bearing surface.

In a further embodiment of the invention, the circumferential area is about up to half the circumference of the annular face.

This measure has the advantage that it is able to create a relatively large surface of lateral bearing in the area of the point, while at the same time however at least half the circumference of the entire annular face is available as a surface for forming the shoulder.

In a further embodiment of the invention, the projecting point has a contour curved in an arc shape.

This measure has the advantage that in certain spatial and pivoting directions a permanently large bearing surface remains available, which, as a result of the gentle contour, slides atraumatically along the tissue surfaces to which the point is applied.

This embodiment can also be used to provide a relatively large cutting surface, so that the distal end of the guide sleeve can have a cutting action, for example in order to cut out a suitable seat in an unstructured, uncontrolled cartilage surface.

In a further embodiment of the invention, the point is formed by a bevelled cut at the distal end of the guide sleeve.

This measure has the advantage that not only can the desired geometry be easily attained, it is also possible to create gentle sliding and bearing surfaces.

In a further embodiment of the invention, the point is designed as a needle-like point projecting from the annular face.

This measure has the advantage that a needle-like point can initially be pushed in to obtain a first position, after which the guide sleeve however can be tilted into another position, and only after this is the suture thread anchor applied.

In a further embodiment of the invention, the needle-like point has an outermost tip which lies on an outer circumferential line of the guide sleeve.

This embodiment has the advantage that the tip comes to lie on the outermost edge of the guide sleeve, thus resulting in a very wide manoeuvring range inside the circumference of the guide sleeve.

In a further embodiment of the invention, an anchor rotator is provided whose shank can be pushed through the guide sleeve.

This measure known per se completes the instrumentation for performing such operations.

In a further embodiment of the invention, the external diameter of the shank of the anchor rotator corresponds to the clear internal diameter of the guide sleeve.

This measure advantageously contributes to ensuring correct and targeted guidance during fitting of the suture thread anchor.

In a further embodiment of the invention, a suture thread anchor is fitted in the anchor rotator.

This measure has the advantage of making available a set of instruments adapted to one another for fitting the suture thread anchor.

It will be appreciated that the aforementioned features and the features still to be explained below can be used not only in the respectively stated combination, but also in other combinations or in isolation, without departing from the scope of the present invention.

### BRIEF DESCRIPTION OF THE DRAWINGS

Illustrative embodiments of the invention are explained and described in more detail below with reference to the attached drawings, in which:

FIG. 1 shows a longitudinal section through an anchor rotator,

FIG. 2 shows a greatly enlarged cross section of the distal end of the anchor rotator into which a suture thread anchor is to be fitted,

FIG. 3 shows a corresponding suture thread anchor for inserting into the distal end of the anchor rotator,

FIG. 4 shows a first illustrative embodiment of a guide sleeve according to the invention with a point which extends about almost half the circumference of the front annular face, and the anchor rotator shown in FIG. 1 can be pushed with a matching fit through the guide sleeve,

FIG. 5 shows a side view of the distal end area of the guide sleeve from FIG. 4,

FIG. 6 shows a cross section along line VI-VI from FIG. 5,

FIG. 7 shows a longitudinal section through a device according to the invention in the area of the distal end area, i.e. of the guide sleeve from FIG. 4, into which an anchor rotator from FIG. 1 is inserted, the anchor shown in FIG. 3 being inserted into the distal end area of said anchor rotator, and the suture thread also being shown here, this device being applied on a glenoid margin during fixation of the labrum/ligament complex,

FIG. 8 shows a view, corresponding to FIG. 4, of a further illustrative embodiment of a guide sleeve with a needle-like projecting point,

FIG. 9 shows a side view corresponding to FIG. 5, and

FIG. 10 shows a cross section, corresponding to FIG. 6, through this illustrative embodiment of a guide sleeve,

FIG. 11 shows a view corresponding to the view in FIG. 7, and here the anchor rotator from FIG. 1, with the suture thread anchor from FIG. 3 fitted in it, is shown in the illustrative embodiment of a guide sleeve with a needle-like point.

### DETAILED DESCRIPTION OF PREFERRED EMBODIMENTS

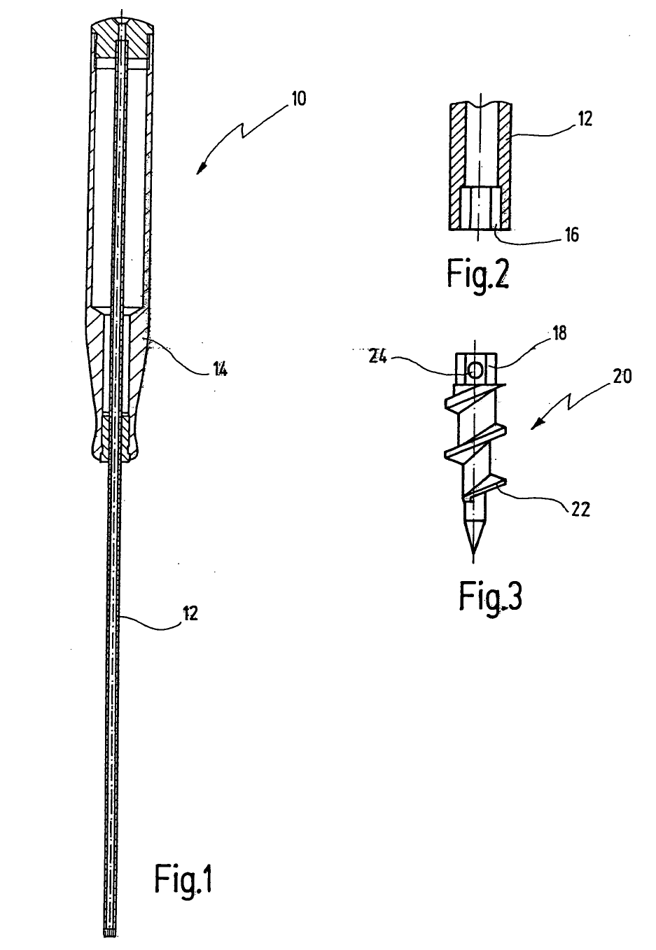

An anchor rotator 10 shown in FIG. 1 has an elongate shank 12 and a grip 14.

At the distal end, shown enlarged in FIG. 2, the shank 12 is provided with an internal hexagon socket 16 into which an external hexagon 18 of a suture thread anchor 20, shown in FIG. 3, can be inserted with an exact fit.

On its outside, the suture thread anchor 20 has a thread 22 and, at its proximal end, it is provided with an eyelet 24 through which a suture thread (not shown here) can be pulled.

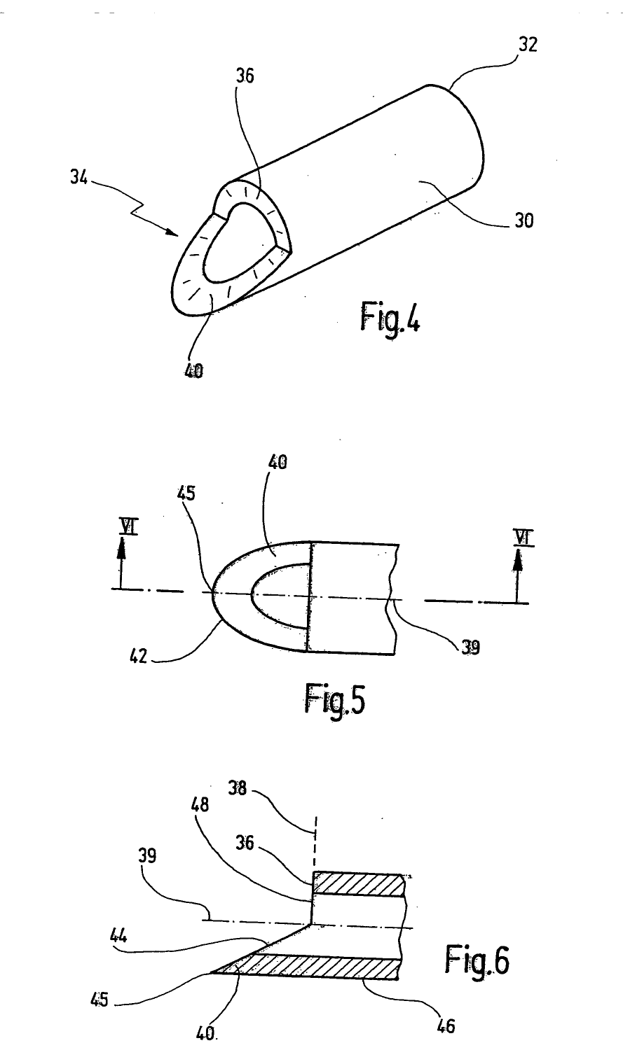

FIG. 4 shows a first illustrative embodiment of a guide sleeve 30, to be precise only in the area of its distal end.

The guide sleeve 30 consists of a tube 32 which, at its proximal end, is provided with a laterally protruding grip (not shown here for the sake of clarity). The distal end 34 has a front annular face 36 whose plane 38 extends approximately perpendicular to the longitudinal axis 39 of the guide sleeve 30, as can be seen in particular from FIGS. 5 and 6.

A point 40 with an approximately arc-shaped contour 42 projects from the front annular face 36. The circumferential extent of the point 40 is such that it takes up about half the circumference of the front annular face 36.

As can be seen from FIG. 6 in particular, the point has been formed in such a way that a bevelled cut 44 has been applied.

The outermost point 45 lies here on an outer circumference line 46 of the tube 32. The bevelled cut 44 is so configured that it encloses an angle of about 30° to the longitudinal axis 39. The angle of the bevelled cut 44 and also the circumferential extent of the point 40 can vary, although it must always be ensured that a shoulder 48 is formed between the point 40 and the front annular face 36.

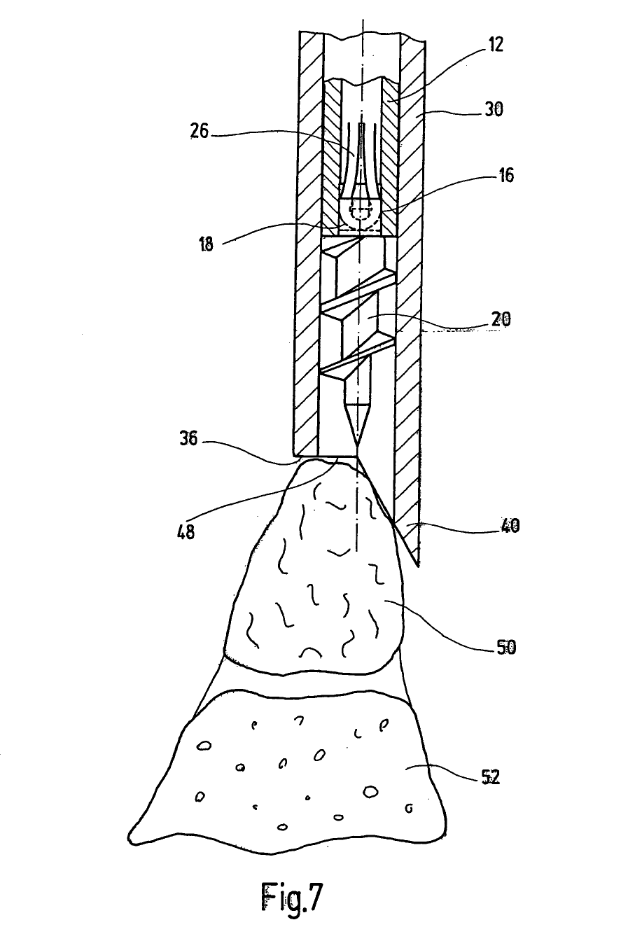

FIG. 7 shows how the device according to the invention is used when fitting a suture thread anchor 20 with which a glenoid margin 50 consisting of cartilage and tendon, which has detached from the bone 52 of the glenoid margin 50, can be fixed back in position. FIG. 7 shows a situation where the assembly comprising guide sleeve 30, anchor rotator 10 and suture thread anchor 20 with suture thread 26 is placed on the glenoid margin 50. By means of the shoulder 48, this assembly can be applied with a secure fit, although the entire device can still be positioned, for example by pivoting it to the left or right in the plane of the drawing or by pivoting it towards or away from the person looking at this drawing. In this way, an ideal position can be found for positioning the suture thread anchor 20.

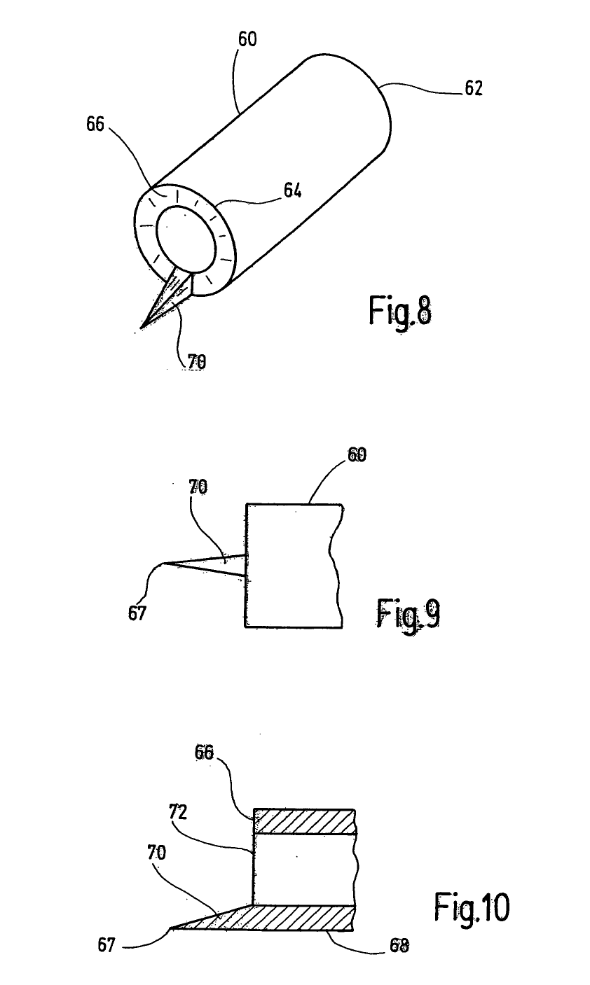

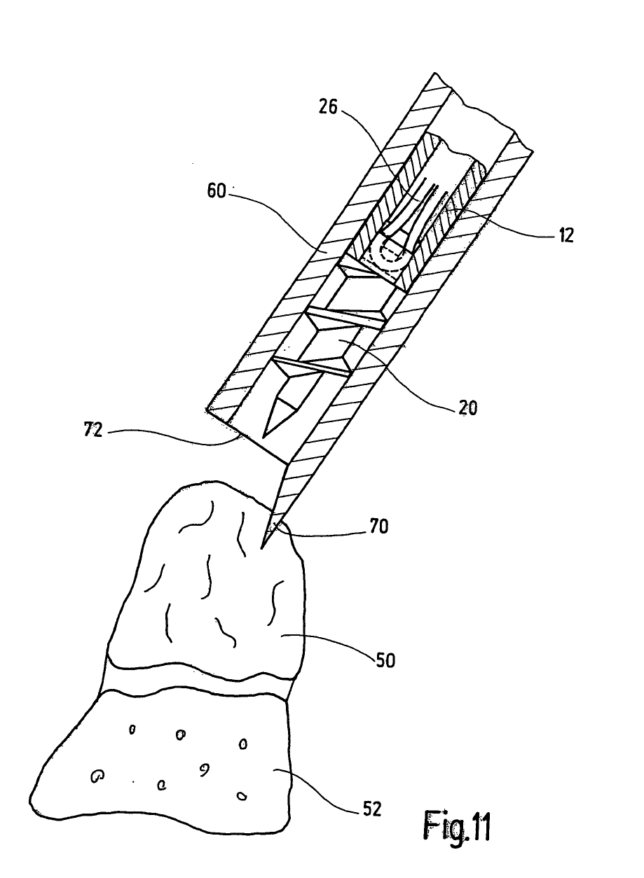

In a further illustrative embodiment shown in FIGS. 8 to 11, a guide sleeve 60 again consists of a tube 62 at whose distal end 64 a front annular face 66 is formed, which, as has been described above, likewise extends approximately at right angles to the longitudinal axis.

A needle-like point 70 projects from the front annular face 66, and the outermost tip 67 of this point lies along a circumferential line 68 of the tube 62.

Here too, a shoulder 72 is created between the point 70 and the distal end 64, while here a relatively large part of the circumference of the front annular face 66 is available as a bearing surface.

The point 70 has an approximately pyramidal shape, with an outer surface extending along the outer circumference face of the tube 62.

FIG. 11 shows a situation in which the anchor rotator 10, with suture thread anchor 20 fitted therein and engaged suture thread 26, has, in turn, been pushed into the guide sleeve 60.

Here, the needle-like point 70 can first be pushed into the corresponding tissue, for example a tendon or a cartilage, or into a bone, and thereafter the exact orientation can be selected. By pushing it in farther, the device comes to lie securely via the shoulder 72 between point 70 and front annular face 66.

1. A device for introducing a suture thread anchor into a bone, comprising
a hollow guide sleeve through which a suture thread anchor can be passed, said hollow guide sleeve having a proximal end and a distal end,
said distal end of said guide sleeve having a front annular face,
a single point projecting distally from said front annular face of said guide sleeve,
wherein a shoulder is formed between said distally projecting single point and a remaining part of said front annular face.

2. The device of claim 1, wherein said front annular face extends approximately perpendicular to a longitudinal axis of said guide sleeve.

3. The device of claim 1, wherein said projecting point projects from over a circumferential area of said front annular face.

4. The device of claim 3, wherein said circumferential area is about to half of a circumference of said front annular face.

5. The device of claim 1, wherein said projecting point has a contour curved in an arc shape.

6. The device of claim 5, wherein said point is formed by a bevelled cut at said distal end of said guide sleeve.

7. The device of claim 1, wherein said point is designed as a needle-like point projecting from said front annular face.

8. The device of claim 7, wherein said needle-like point having an outermost tip which lies on an outer circumferential line of said guide sleeve.

9. The device of claim 1, wherein an anchor rotator is provided, whose shank can be guided through said hollow guide sleeve.

10. The device of claim 9, wherein an external diameter of said shank of said anchor rotator approximately corresponds to a clear internal diameter of said guide sleeve.

11. The device of claim 10, wherein said suture thread anchor is fitted in said anchor rotator.
